(19) United States
(12) Patent Application Publication
(10) Pub. No.: US 2016/0143561 A1
(43) Pub. Date: May 26, 2016

(54) SELF-CONTAINED, PORTABLE H2/CO2 (AIR) RATIO APPARATUS

(71) Applicant: ONY, Inc., Amherst, NY (US)

(72) Inventor: Edmund A. Egan, II, Amherst, NY (US)

(73) Assignee: ONY, Inc.

(21) Appl. No.: 14/862,695

(22) Filed: Sep. 23, 2015

Related U.S. Application Data
(60) Provisional application No. 62/054,766, filed on Sep. 24, 2014.

(51) Int. Cl. A61B 5/08; A61B 5/00; A61B 5/097; A61B 5/087

(52) U.S. Cl. CPC A61B 5/082; A61B 5/087; A61B 5/742; A61B 5/097

(57) ABSTRACT

A self-contained, portable H₂/CO₂ (air) ratio apparatus has an exhaled breath system, an air analyzing chamber, a hydrogen concentration analyzer, a carbon dioxide (air) concentration analyzer, an air outlet, a computational analyzer and a display unit. The display unit can be a part of the self-contained, portable H₂/CO₂ (air) ratio apparatus, remotely a part of the self-contained, portable H₂/CO₂ (air) ratio apparatus, or combinations thereof. The apparatus can assist in the diagnosis of a neonatal patient developing necrotizing enterocolitis and/or lactose intolerance.

### CROSS REFERENCE TO RELATED APPLICATIONS

This application claims priority from U.S. Provisional Patent Application No. 62/054,766, filed on Sep. 24, 2014, which is hereby incorporated by reference in its entirety.

### TECHNICAL FIELD

This application is directed to a self-contained, portable H₂/CO₂ (air) ratio apparatus used to monitor the size of the bacterial load of the gut in a patient, in particular a neonatal patient, that has a significant chance of developing Necrotizing Enterocolitis or, alternatively, is lactose intolerant.

### BACKGROUND ART

A generic explanation of Necrotizing Enterocolitis (NEC) in the Premature Infant is expressed in Adv Neonatal Care. 2011 Jun; 11(3); 155-166. In that article Gregory et al. wrote, “Necrotizing enterocolitis (NEC) is the most common life-threatening gastrointestinal emergency experienced by premature infants cared for in the Newborn Intensive Care Unit (NICU). It is a devastating gastrointestinal disease that is associated with severe sepsis, intestinal perforation, and significant morbidity and mortality. The incidence of NEC is inversely correlated to gestational age and birth weight. Low birth weight, premature infants are affected at a prevalence as high as 15% of all infants cared for in the NICU. More than 11% of infants born at birth weights below 750 grams will develop NEC. Though the majority of NEC cases are treated medically, an estimated 20 to 40% of infants will require urgent surgical intervention including exploratory laparotomy, bowel resection, and ostomy. The case fatality rate associated with surgical intervention is as high as 50%, and is highest among the smallest and most premature infants. Infants who survive are prone to short bowel syndrome, parenteral nutrition-associated cholestasis, prolonged neonatal hospitalization, significantly impaired growth, and poor long-term neurodevelopment. These infants require long term nursing care that is complicated and costly. The yearly additional hospital charges for NEC in the United States are estimated in excess of $6.5 million. Epidemiologic risk factors and clinical predictors have been explored; however, only prematurity has been identified as a consistent risk factor associated with NEC.

NEC is one of the major unsolved problems associated with premature birth. Abnormal bacterial colonization of the immature gut is a significant risk factor identified with NEC. Prior to birth, the newborn intestine is essentially sterile. At birth, the newborn intestine is inoculated and under ideal circumstances, a normal intestinal immune defense system develops. The pathophysiology that underpins NEC leads to atypical immunity and inflammatory response. Furthermore, this evidence has been corroborated by the fact that one of the consistently identified protective effects against NEC is feeding with breast milk. Breastfed infants have a lower incidence of NEC than formula-fed infants. This finding has been explained by the notion that breastfeeding facilitates colonization of a balanced, non-pathogenic flora in the gut that helps prevent bacterial overgrowth, whereas formula feeding promotes abnormal, pathogenic bacterial growth. Aberrant colonization and bacterial overgrowth produces food-induced toxic by-products including unique microbial molecular patterns that are capable of altering the epithelial barrier and triggering an inflammatory cascade of the immature intestinal innate immune system. This specific inflammatory cascade has been hypothesized to be central in triggering the onset of pathogenesis of NEC.

The significance of bacterial colonization and importance of balancing harmful and helpful bacteria in the premature gut is a topic of great interest to neonatal researchers and clinicians. This interest has underpinned studies exploring the effect of widespread antibiotics on the incidence of NEC, the role of specific intestinal microbiota in the development of NEC, and ultimately, the potential use of nutritional strategies such as pre- and probiotics for disease prevention. Findings have shown that prolonged duration of initial empirical antibiotic treatment is significantly associated with increased rates of NEC in extremely low birth weight infants, presumably because these drugs disrupt the normal colonization of the neonatal intestinal microbiota. Investigators have also shown that certain microbiota such as Lactobacillus and Bifidobacteria are protective against NEC, while other species such as Enterobacteriaceae, Clostridia, and Staphylococcus are commonly implicated in the pathogenesis of the disease. That said, recent evidence generated on the most technologically advanced platform suggests that no one microorganism is predictive of the disease. Rather, a predominance of Proteobacteria is highly associated with NEC. According to this study, the limited diversity of total bacteria and abundance of pathogenic bacteria may contribute to the susceptibility of the premature infant gut to NEC. Further study on the microbiological aspects of disease holds promise for new knowledge that may result in preventative strategies including the use of selected prebiotics and probiotics.

Clinical presentation of NEC may vary among infants, which presents a challenge to clinicians aiming to diagnose the disease at the earliest and least severe stage of pathogenesis. The disease may present anywhere on the clinical spectrum, ranging from slow and insidious to rapid and progressive. Systemic, intestinal, and radiological signs all play a role in diagnosis, but vary in observed presence and degree of involvement. Assigning disease severity based on staging criteria for NEC is important in the diagnosis and treatment of the disease. Bell Staging has traditionally been the standard in assigning severity of disease to NEC cases.

Dr. Martin Bell proposed the original clinical criteria used to stage NEC cases in 1978. Three stages were outlined to enhance the recognition and diagnosis of NEC, and to provide the most effective treatment for each cohort of patients. The proposed staging criteria have been modified as our understanding of NEC has evolved to incorporate further specificity into each stage of disease. Even with these subsequent modifications, it has recently been suggested that the Bell Staging criteria are outdated as a result of the increase in viability at lower gestational ages, the improvement in clinical management of medical NEC, and the occurrence of other acquired neonatal intestinal diseases that differ from premature infant NEC. Bell Staging, however, continues to be used as the standard of practice to diagnose, stage, and treat NEC in the NICU.

Stage 1, or suspected NEC, includes patients who present with the mildest of symptoms. The diagnosis of NEC is often questionable, and should be suspected after other examinations rule out other gastrointestinal disorders. Systemic manifestations include temperature instability, lethargy, apnea, and bradycardia. The infant may feed poorly, have increasing pregavage residuals, vomit, present with a mildly distended abdomen, or pass stool with occult blood. Bowel loops may be distended on radiographic evaluation with mild ileus. Infants with a suspected NEC diagnosis who have disease that progresses to include the classic radiological sign of pneumatosis intestinalis are classified as Stage II, or proven, NEC cases. This classification of patients present with signs more indicative of NEC than Stage I after other gastrointestinal disorders have been ruled out. Abdominal distention in these patients is marked, and persistent occult or frank blood in the stool may be present. Radiological signs may include pneumatosis intestinalis, persistent or unchanging bowel loops, and the development of portal vein gas.

Advanced NEC cases are designated as Stage III, and includes those patients showing most or all symptoms present in Stages I and II. Stage III infants show a deterioration of vital signs, evidence of septic shock, or marked gastrointestinal bleeding. Bowel necrosis may occur by the time the diagnosis is made, at times requiring surgical intervention. In the advanced stage, pneumoperitoneum may be present on abdominal films in addition to the radiographic signs for Stages I and II. Cases of NEC in which few clinical signs are present before the patient develops pneumoperitoneum are rare; however, these patients are classified as Stage III. Modifications have been made to the Bell Staging criteria by researchers studying NEC aiming to more definitively define the differences in diagnosis stages. [Some researchers] break each stage into two subcategories, and include signs that differentiate between milder and more severe courses of disease. Newly included in the staging criteria are lab values indicative of acidosis, thrombocytopenia, neutropenia, and disseminated intravascular coagulation, in addition to intestinal signs of absent bowel sounds and abdominal tenderness.” That generic overview of NEC has been confirmed in numerous other articles.

In Breath Hydrogen Excretion as a Screening Test for the Early Diagnosis of Necrotizing Enterocolitis, Am J Dis Child. 1989; 143(2): 156-159; Cheu et al. wrote, “We measured breath H₂ excretion in 122 neonates from birth to 1 month of age. The patients weighed less than 2000 g at birth and thus were at risk for developing necrotizing enterocolitis (NEC). Hydrogen excretion was normalized for the quality of the expired air by dividing by the carbon dioxide pressure of the gas sample. The mean (±SD) peak H₂/CO₂ ratio was significantly different between the seven patients who subsequently developed NEC (9.4+2.7 ppm/mm Hg) and the 115 patients who did not (5.0±3.5 ppm/mm Hg). The prevalence of NEC was 5.7% in the present study. Defining a positive test as one with a ratio value of greater than or equal to 8.0 ppm/mm Hg, the resulting screening test had a sensitivity of 86% and a specificity of 90%. The screening test yielded a 33% predictive value of a positive test and a 99% predictive value of a negative test. High H₂ excretion occurred eight to 28 hours before the earliest clinical signs of NEC. Breath H₂ excretion is a simple noninvasive test that may be useful in the management of the premature neonate at risk for the development of NEC.”

In particular Cheu et al. taught “Gas samples were collected from intubated infants through a modified endotracheal tube adaptor that allowed one person to collect exhaled gas in a syringe while another person provided ventilator support by hand at a rate and pressure that simulated that of a respirator. If the patient was not receiving ventilator support from a respirator, a feeding tube . . . was inserted through the nose into the hypopharynx and exhaled gas was collected by coordinating withdrawal with the patient's exhalations. A 60-ml syringe . . . with an attached three-way stopcock . . . was used to collect and store the specimens at room temperature. All specimens were analyzed within 12 hours of collection. Hydrogen content in parts per million was measured on a gas chromatograph (Quintron, Milwaukee). The chromatograph has a sensitivity of less than 3 ppm and an accuracy to within 3 ppm and was calibrated daily using a standard of known concentration. The carbon dioxide pressure (PCO₂) was measured on a CO₂ monitor (Puritan-Bennett, Kansas City, Mo.). Samples were collected daily without regard to the time of feedings since preliminary studies in which infants were tested every half hour through several feeding cycles showed no significant variation because of feeding. . . . All breath H₂ values were divided by the PCO₂ of the sample as a means of standardizing for the quality of the expired gas. The PCO₂ and H₂ concentration of sequential specimens taken from the subject over a short time, by either the same or different individuals, showed coefficients of variation of 40% and 38%, respectively.

Cheu et al.'s method has serious flaws and those flaws were exposed by Young et al. in Biomarkers for Infants at Risk for Necrotizing Enterocolitis: Clues to Prevention?, Pediatr Res. 2009 May; 65(5 Pt 2): 91R-97R. In particular Young et al. wrote, “It was initially concluded that breath H₂ excretion is a simple non-invasive test that may be useful in the management of the premature neonate at risk for the development of NEC. However this test has not received acceptance largely because of technical difficulties in measuring the pressure of exhaled hydrogen, the large variations in food intake, the variability in the time it takes for bacterial colonization, and the poor predictive value for a positive test.”

Measuring H₂ concentration is a standardized process. One of those standardized processes utilizes the above-identified gas chromatograph. A gas chromatograph is appropriate for a laboratory setting when a measurement is required once or twice a day. A gas chromatograph is not appropriate when a measurement has to be repeated numerous times in one day. That is one reason Cheu's method is not practical for monitoring bacteria growth to determine NEC or alternatively, for example, lactose intolerance.

There are many different hydrogen sensor technologies. Hydrogen sensors based on thermoelectric effects, thermal conductivity, catalytic combustion (combustible gas sensors), surface plasmon resonance, both aqueous and solid-state electrochemistry, heated metal-oxide (HMOX) electronic devices, surface acoustic wave or cantilever mechanical devices, optical and electronic effects in carbon nanotubes, optical and electronic palladium film technology, and other techniques have been reported for various applications.

Hydrogen sensors are needed that can operate at temperatures from −30 to 1000° C., (preferably for this invention room temperature and patient temperature) and this results in the use of unique designs and new materials. Some applications demand extremely high selectively over a wide concentration range from 10 ppm to 100% in many different background gases. For many widespread applications, a sensor with simplicity and low cost as well as small size and minimal power consumption is desired. Electrochemical approaches to sensing H₂ can provide one of the lowest power approaches for gas monitoring combined with good analytical performance.

The amperometric gas sensor, or AGS, combines versatility, sensitivity, and ease of use in common gas detection situations with a relatively low cost and, recently, with the possibility for miniaturization. The simplest amperometric cell consists of two electrodes, i.e., a working electrode and a counter electrode, and the electrolyte solution in which the two electrodes are immersed. The amperometric gas sensor is operated under an externally applied voltage, which drives the electrode reaction in one direction. This two electrode detection principle presupposes that the potential of the counter electrode remains constant. A potentiostat is usually used to fix this working electrode potential. In reality, the surface reactions at each electrode cause them to polarize, and significantly limit the concentrations of reactant gas they can be used to measure. Therefore, in practice many amperometric sensors have a more complicated physical configuration and, specifically, are built according to a three-electrode scheme. In three electrode designs, it is still the current between working and counter electrodes that is measured and the reference electrode maintains the thermodynamic potential of the working electrode constant during sensing. Since the reference electrode is often shielded from reactions, it maintains a constant thermodynamic potential during sensing. The inlet can be simple diffusion or aided by a small air pump that transports the sample to the gas porous membrane through which the analyte diffuses/permeates to the sensing electrode. There is also an H₂ AGS in a thin film “fuel-cell” configuration. In this variant of AGS, the current generated by reaction of hydrogen at the sensing or working electrode is measured as the sensor signal and it can be measured at a fixed or variable electrode potential. Electroactive analyte, i.e. the H₂ participating in electrochemical reaction, diffuses from the surrounding gas to the cell, through the porous layers, and dissolves in the electrolyte, through which it proceeds to diffuse to the working electrode surface. The reaction rate, reflected by the current at the sensing electrode, can be limited by the rate of reaction at the surface or the rate of diffusion of the H₂ to the electrode surface. If operated under diffusion-limited conditions, the current is proportional to the concentration of the analyte in the surrounding gas. Application of Faraday's law relates the observed current (sensor signal) to the number of reacting molecules (concentration) by

I=nFQC

where I is current in coulombs/s, Q is the rate of gas consumption in m³/s, C is the concentration of analyte in mol/m³, F is Faraday's constant (9.648×10⁴ coulombs/mol), and n is the number of electrons per molecule participating in the reaction. In general, the Nafion electrolyte H₂ sensor electrode processes include the anode reaction

H₂(g)→2H⁺+2e⁻

and the cathode reduction reaction

½O₂+2H⁺+2e⁻→H₂O (liq)

Under short-circuit conditions, reaction (H₂(g)→2H⁺+2e⁻) occurs at the sensing or working electrode, WE, whereas reaction (½O₂+2H³⁰+2e⁻→H₂O (liq)) occurs at the counter, or CE, electrode. Simultaneously, the protons move toward the counter electrode through the proton conducting electrolyte. This process results in a flow of an equivalent number of electrons in an external electrical circuit. The anodic oxidation reaction of hydrogen is often limited by the diffusion process to the sensing electrode by the sensor design. The number of protons produced is proportional to the hydrogen concentration. Since, the number of H₂ molecules reaching the WE are limited by diffusion of H₂ from the air to the WE surface, the external current is proportional to the hydrogen concentration in the gas phase, which can be derived from combining Fick's law and Faraday's law, correlating the flux/number, J_{H}_{₂}, of hydrogen molecules being pumped per second to the current I:

J_{H}_{₂}=I/2q

where I is current in coulombs/s and q is the electric charge of an electron (1.6×10⁻¹⁹ coulombs). The flux of hydrogen diffusing through the aperture of an amperometric sensor is given by Fick's first law:

J_{H}_{₂}=A·D_{H}_{₂}[∂P_{H}_{₂}/∂x]

where A is the area of the diffusion barrier in m², D is the diffusion coefficient in m²/s, P_{H}_{₂} is the hydrogen concentration in mol/m³, and x is the thickness of the barrier in m. Thus, the two immediately identified equations can be rearranged to give the current:

I=2qAD_{H}_{₂}[∂P_{H}_{₂}/∂x]

The mechanism of a hydrogen gas sensor can be described in four steps: (1) the gas (H₂) diffuses through the gas-diffusion barrier to the electrode and is then adsorbed on the sensing electrode H2(ads), (2) the electrochemical reaction (H₂(g)→2H⁺+2e⁻) occurs with electron transfer and generation of H⁺, (3) the protons move toward the counter electrode through the proton-conducting membrane, (4) the resulting reaction (½O₂+2H⁺+2e⁻→H₂O (liq)) occurs with electron transfer, and the product desorbs from the counter electrode and diffuses away. The electronic charge is transferred to or from the electrodes in steps (2) and (4) and is observed as a current through the external circuit.

Another method to measure H₂ concentration is a biosensor. The biosensor is generally defined as an analytical device which converts a biological response into a quantifiable and processable signal. In biosensing, the measurement of electrical properties for extracting information from biological systems is normally electrochemical in nature, whereby a bioelectrochemical component serves as the main transduction element. Although biosensing devices employ a variety of recognition elements, electrochemical detection techniques use predominantly enzymes. This is mostly due to their specific binding capabilities and biocatalytic activity. Other biorecognition elements are e.g. antibodies, nucleic acids, cells and micro-organisms. An immunosensor uses antibodies, antibody fragments or antigens to monitor binding events in bioelectrochemical reactions. Typically in (bio-)electrochemistry, the reaction under investigation would either generate a measurable current (amperometric), a measurable potential or charge accumulation (potentiometric) or measurably alter the conductive properties of a medium (conductometric) between electrodes. Amperometric devices are a type of electrochemical sensor, since they continuously measure current resulting from the oxidation or reduction of an electroactive species in a biochemical reaction. Clark oxygen electrodes perhaps represent the basis for the simplest forms of amperometric biosensors, where a current is produced in proportion to the oxygen concentration. This is measured by the reduction of oxygen at a platinum working electrode in reference to a Ag/AgCl reference electrode at a given potential. Typically, the current is measured at a constant potential and this is referred to as amperometry. The above-identified devices can monitor and measure hydrogen.

Other electrochemical hydrogen sensors are disclosed in Korotcenkov et al.'s Review of Electrochemical Hydrogen Sensors, Chem. Rev. 2009, 109(s) 1402-1433, which is hereby incorporated by reference to disclose alternative hydrogen sensors.

Methods to measure CO₂ are also well known. Carbon dioxide and other gases consisting of two or more dissimilar atoms absorb infrared (IR) radiation in a characteristic, unique manner. Such gases are detectable using IR techniques. Water vapor, methane, carbon dioxide, and carbon monoxide are examples of gases that can be measured with an IR sensor.

IR sensing is the most widely applied technology for CO₂ detection. IR sensors have many benefits over chemical sensors. They are stable and highly selective to the measured gas. They have a long lifetime and, as the measured gas doesn't directly interact with the sensor, IR sensors can withstand high humidity, dust, dirt, and other harsh conditions.

The key components of an IR CO₂ detector are a light source, a measurement chamber, an interference filter, and an IR detector. IR radiation is directed from the light source through the measured gas to the detector. A filter located in front of the detector prevents wavelengths other than that specific to the measured gas from passing through to the detector. The light intensity is detected and converted into a gas concentration value.

In reality, gases do not behave exactly like ideal gases, but the approximation is often used to describe the behavior of real gases. The ideal gas law relates the state of a certain amount of gas to its pressure, volume, and temperature, according to the equation:

pV=nRT where

p=pressure [Pa]

V=volume of the gas [m3]

n=amount of gas [mol]

R=universal gas constant (=8.3145 J/mol K)

T=temperature [K]

Most gas sensors give out a signal proportional to the molecular density (molecules/volume of gas), even though the reading is expressed in parts per million (volume/volume). As the pressure and/or temperature changes, the molecular density of the gas changes according to the ideal gas law. The effect is seen in the ppm reading of the sensor.

The molecules of a gas mixture exist in the same system volume (V is the same for all gases) at the same temperature. The ideal gas law can be modified to:

V=(n_{gas1}+n_{gas2}+n_{gas3}+ . . . n_{gasn})*RT/V where

n_{gas1}=amount of gas 1 [mol]

n_{gas2}=amount of gas 2 [mol], etc. and

P=P_{gas1}+P_{gas2}+P_{gas2}+ . . . P_{gasn} where

p=total pressure of the gas mixture

p_{gas1}=partial pressure of gas 1

P_{gas2}=partial pressure of gas 2, etc.

The latter equation is called Dalton's Law of Partial Pressure. It states that the total pressure of a gas mixture is the sum of the partial pressures of all the component gases in the mixture.

Carbon dioxide is measured at a wavelength that is relatively transparent to water and is known to those having ordinary skill in the art.

The problems with Chau et al.'s H₂/CO₂ ratio method are well known and that ratio method has not been utilized for a while.

### BRIEF SUMMARY OF THE INVENTION

The claimed invention is directed to a self-contained, portable H₂/CO₂ (air) ratio apparatus that can repeatedly measure the amount of (a) hydrogen in an exhaled breath, and (b) carbon dioxide in the exhaled breadth. It is widely accepted that 5% of an exhaled breadth is normally carbon dioxide. The ability and capability to repeatedly take such measurements can assist a medical provider expeditiously determine if a patient has an increased chance of developing Necrotizing Enterocolitis or lactose intolerance.

This self-contained, portable apparatus capitalizes on a law of nature that hydrogen is released by bacteria. The amount of hydrogen released is a measure of the number of the bacteria in the bowel. Rapid increases in hydrogen and/or high levels of hydrogen production indicate a condition called bacterial overgrowth of the bowel. If gut bacteria are exposed to unabsorbed food that has not yet had a chance to completely traverse the intestine and be fully digested and absorbed they will proliferate. Some of the hydrogen produced by the bacteria, whether in the small intestine or the colon, is absorbed into the blood flowing through the wall of the small intestine and colon. The hydrogen-containing blood travels to the lungs where the hydrogen is released and exhaled in the breath where it can be measured. This invention is not directed to that law of nature; instead this invention is directed to a self-contained, portable apparatus that can capitalize on that law of nature. As clearly identified above, there are numerous methods to measure hydrogen and carbon dioxide. Applicant is not attempting to limit others from using those prior laboratory setting protocols. Instead Applicant is concentrating exclusively on a self-contained, portable apparatus to conduct those measurements.

### BRIEF DESCRIPTION OF THE DRAWINGS

FIG. 1 is schematic of a self-contained, portable H₂/CO₂ (air) ratio apparatus;

FIG. 2 is an embodiment of an exhaled breath system; and

FIG. 3 is an alternative view of the exhaled breath system.

### DETAILED DESCRIPTION OF EMBODIMENTS

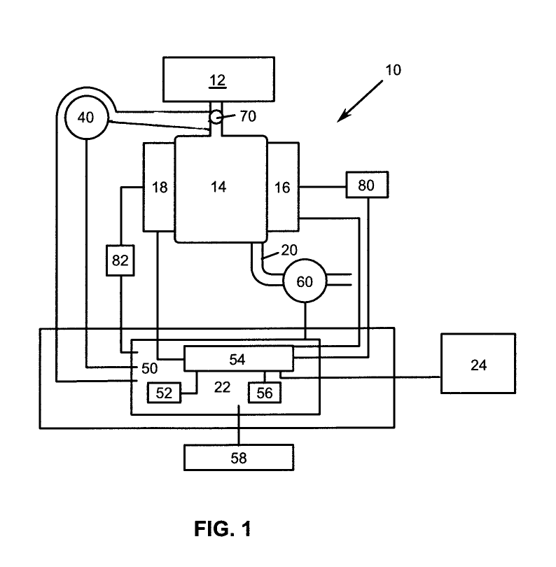

A self-contained, portable H₂/CO₂ (air) ratio apparatus 10 is illustrated, in a flow chart form, at FIG. 1. The self-contained, portable H₂/CO₂ (air) ratio apparatus 10 has an exhaled breath system 12, an air analyzing chamber 14, a hydrogen concentration analyzer 16, a carbon dioxide (air) concentration analyzer 18, an air outlet 20, a computational analyzer 22 and a display unit 24. The display unit 24 can a part of the self-contained, portable H₂/CO₂ (air) ratio apparatus 10, remotely a part of the self-contained, portable H₂/CO₂ (air) ratio apparatus 10; or combinations thereof

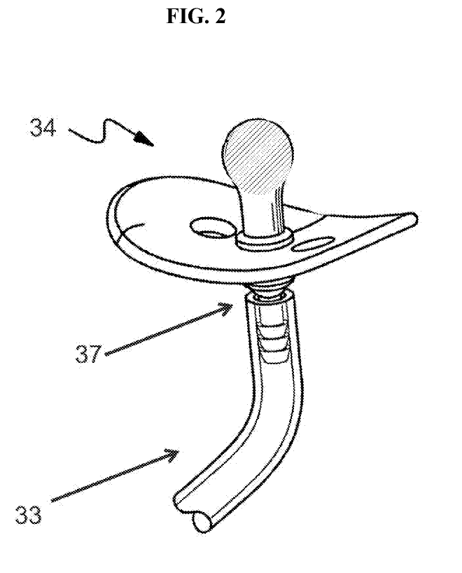

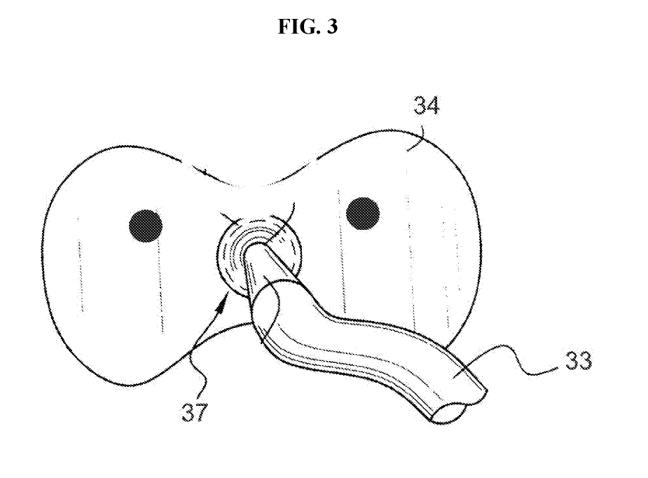

Turning to FIGS. 2 and 3, the exhaled breath system 12 has a pacifier 34, an exhaled delivery tube 33 (for example and not limited to a polyvinylchloride tube, 5fr or larger conventional “feeding tube”) and a lock collar 37. By using the pacifier 34, the distal orifice of the exhaled delivery tube 33 is placed in the patient's pharynx area 4 while assuming that the distal orifice of the exhaled delivery tube 33 is not obstructed by the patient's tongue, buccal mucosa or pharyngeal wall, and also inhibiting that the distal end of the exhaled delivery tube 33 from abrading or piercing the patient's airway tissues. The pacifier 34 may be fashioned from a material that allows the patient to comfortably bite down on the pacifier 34. As illustrated the exhaled breath system 12 is illustrated at FIGS. 2 and 3 is comprised of several distinct parts, each of which may be selectively disengaged from the others. The invention, however, need not be formed from selectively disengagable parts, and instead may be an integrated set of parts that are not selectively disengageable.

The proximal end of the exhaled delivery tube 33 interconnects to the air analyzing chamber 14. Positioned between the lock collar 37 and the proximal end of the exhaled delivery tube 33 is an aspirator or pump (manual or motorized) 40 that pulls the exhaled breath into the exhaled delivery tube 33. The aspirator 40 is applied for a predetermined time period, for example 30 to 60 seconds, in order to collect a predetermined, and sufficient quantity of an exhaled breadth, for example, 10-100 mL.

The collected exhaled breath is positioned in the air analyzing chamber 14 by pushing the air analyzing chamber's residual air through the air outlet 20. The air analyzing chamber 14 has an exterior surface that can be dynamic like a balloon or static like a metal or hard plastic container, which permits the collected exhaled breadth to permeate or is directed (for example through an opening(s)) to the hydrogen concentration analyzer 16, and the carbon dioxide (air) concentration analyzer 18. Once the collected exhaled breath is positioned in the air analyzing chamber 14, the collected exhaled breath is analyzed by the hydrogen concentration analyzer 16, and the carbon dioxide (air) concentration analyzer 18. Both analyzers 16, 18 are preferably manufactured by Sensorcon, Inc. of Buffalo, N.Y. The hydrogen concentrator analyzer 16 is similar to any of the above-identified hydrogen measurement devices with the understanding that the preferred embodiment is an amperometric type electrochemical sensor having a range of 0 to 1999 parts per million (ppm), a resolution of 1 ppm, an accuracy of +/−10% of reading or +/−2 ppm (whichever is higher), has a temperature range of −4 to +122° F. (−20 to +50° C.), can measure the H₂ as low at 1 ppm and a humidity range of 20 to 90% relative humidity continuous. Similarly, the carbon dioxide (air) concentration analyzer 18 is similar to any of the above-identified carbon dioxide measurement devices with the understanding that the preferred embodiment is an infrared version that can measure the carbon dioxide concentration to 0.1%. That value is significant because if the carbon dioxide concentration is less than 5,000 ppm; then the collected exhaled breadth is a deemed an inadequate sample and the results are identified appropriately. It is understood that both analyzers 16, 18 should operate properly when at ambient room temperature and the breadth is at the patient's temperature.

Both analyzers 16, 18 are interconnected to the air analyzing chamber 14. The interconnection can be remote, for example, through conduits; or integrated with the air analyzing chamber's side surface as illustrated at FIG. 1. In either version, the respective analyzers 16, 18 measure the concentration of, respectively, hydrogen and carbon dioxide from the collected breadth. Each respective analyzers' 16, 18 measurement is an electronic signal in units of parts per million. Those measurements are respectively transmitted to the computational analyzer 22.

The computational analyzer 22 can have a central processing unit 50 having a clock system 52, a calculation system 54 and a data-storage system 56; and an input device 58, and the computational analyzer 22 is interconnected to a display unit 54. Prior to inserting the exhaled breath system 12 into the patient's mouth for the first time, a medical provider enters the patient's name, patient's identification number, patient's date of birth, and any other relevant identification information through the input device 58 into the central processing unit 50. When the exhaled breath system 12 is positioned into the patient's mouth; the medical provider indicates to the computational analyzer 22 that a breath measurement will immediately or shortly occur. When the exhaled breath is collected, the hydrogen concentration analyzer 16, and the carbon dioxide (air) concentration analyzer 18 measure, respectively the hydrogen and the carbon dioxide in the collected exhaled breath. Those first measurements are transmitted to the calculation system 54 and the data-storage system 56. The data storage system 56 confirms the time and enters into a conventional memory unit the first measurement's time, the first hydrogen measurement, and the first carbon dioxide measurement. Simultaneously, soon thereafter or prior, the calculation system 54 calculates a first H₂/CO₂ ratio value for the first measurements. The first H₂/CO₂ ratio value is (a) calculated by dividing the first hydrogen measurement by the first carbon dioxide measurement; and (b) associated with the above-identified first measurement's time, the first hydrogen measurement, and the first carbon dioxide measurement in the data storage system 56 to obtain a first result.

The first result, along with the patient's identification information is transmitted to the display unit 54. The display unit 54 can be a part of the self-contained, portable H₂/CO₂ (air) ratio apparatus 10, a remote part of the self-contained, portable H₂/CO₂ (air) ratio apparatus 10, or combinations thereof.

Additional hydrogen measurements, carbon dioxide measurements, H₂/CO₂ ratio values and measurement times are made, recorded and displayed. The additional measurements and ratio values are displayed with prior measurements associated with the patient.

In a preferred embodiment, the self-contained, portable H₂/CO₂ (air) ratio apparatus 10 is used for one patient; and then returned to the manufacturer for calibration of each component. Alternatively, the apparatus 10 can have a hydrogen auto calibration system 80 and a carbon dioxide auto calibration system 82 to calibrate, respectively the hydrogen concentration analyzer 16, and the carbon dioxide (air) concentration analyzer 18.

If the aspirator 40 is automated, the aspirator 40 is preferably interconnected to the computational analyzer 22, wherein the computational analyzer 22 is connected to a control circuit switch board that controls the duration and power of the negative pressure generated by the aspirator. Likewise, the air outlet 20 can have a valve 60. The valve 60, like the aspirator, can be manually or mechanically controlled. If mechanically controlled, the valve 60, like one version of the aspirator, is interconnected to the control circuit switch board that controls the duration that the valve is open or closed.

Alternatively, the exhaled breath system 12 can have a breathing sensor 70. The breathing sensor 70, such as a flow transducer or similar flow sensing element, situated within or near the distal orifice of the exhaled delivery tube 33. The breathing sensor 70 merely identifies when the patient is exhaling, inhaling or in transition thereof. The breathing sensor is interconnected to the aspirator 40 and possibly the control circuit switch board to assist in determining at which instant the aspirator 40 should initiate its negative pressure to collect the patient's exhaled air.

Portable means that the self-contained, portable hydrogen/carbon dioxide ratio apparatus can be readily moved from a hospital room to another hospital room within or under 30 minutes.

In addition, self-contained, portable H₂/CO₂ (air) ratio apparatus 10 can be packaged in a kit or positioned on a cart.

The present disclosure contemplates that many changes and modifications may be made. Therefore, while certain embodiments have been illustrated and described, and a number of alternatives discussed, persons skilled in this art will readily appreciate that various additional changes and modifications may be made without departing from the scope of the invention, as defined and differentiated by the following claims.

1. A hydrogen/carbon dioxide ratio apparatus comprising:
a self-contained, portable hydrogen/carbon dioxide ratio unit having:
an exhaled breath system having a pacifier, an exhaled delivery tube having a length that allows the distal orifice of the exhaled delivery tube to be positioned in the patient's pharynx area, and an aspirator to create a negative pressure in the exhaled deliver tube to collect the patient's exhaled breath into the exhaled delivery tube;
an air analyzing chamber interconnected to a hydrogen concentration analyzer, a carbon dioxide concentration analyzer, and an air outlet, the air analyzing chamber receives the collected patient's exhaled breath so (a) the hydrogen concentration analyzer can measure the amount of hydrogen in the collected air breath and (b) the carbon dioxide concentration analyzer can measure the amount of carbon dioxide in the collected air breath; and
a computational analyzer (a) receives the hydrogen measurement and the carbon dioxide measurement, (b) determines whether the collected air breath is an adequate specimen to retain the hydrogen measurement and the carbon dioxide measurement, (c) only when the collected air breath is adequate, calculates a hydrogen/carbon dioxide ratio, and records the hydrogen measurement, the carbon dioxide measurement, the calculated hydrogen/carbon dioxide ratio and time the measurements occurred; and
a display unit (a) receives the hydrogen measurement, the carbon dioxide measurement, the calculated hydrogen/carbon dioxide ratio and time the measurements occurred, and (b) presents the hydrogen measurement, the carbon dioxide measurement, the calculated hydrogen/carbon dioxide ratio and time the measurements occurred on a screen.

2. The apparatus of claim 1, further comprising a hydrogen auto-calibration unit for the hydrogen concentration analyzer.

3. The apparatus of claim 1, further comprising a carbon dioxide auto-calibration unit for the carbon dioxide concentration analyzer.

4. The apparatus of claim 1, wherein the aspirator is mechanically controlled by the computational analyzer.

5. The apparatus of claim 1, wherein the exhaled breath system has a flow sensor that indicates an exhaled breath.

6. The apparatus of claim 5, wherein the flow sensor interconnects to the computational analyzer and computational analyzer initiates the aspirator to create negative pressure in the exhaled breath system when the flow sensor indicates an exhaled breath.

7. The apparatus of claim 1, wherein the air outlet has a valve.

8. The apparatus of claim 1, wherein the valve is mechanically controlled by the computational analyzer.

9. The apparatus of claim 1, wherein the self-contained, portable hydrogen/carbon dioxide ratio apparatus is used to assist in diagnosing necrotizing enterocolitis.

10. The apparatus of claim 1, wherein the self-contained, portable hydrogen/carbon dioxide ratio apparatus is used to assist in diagnosing lactose intolerance.

11. The apparatus of claim 1, wherein the aspirator creates a sufficient negative pressure in the exhaled breath system to collect 10-100 mL of the exhaled breath.

12. The apparatus of claim 1, wherein the aspirator creates a sufficient negative pressure for 30 to 60 seconds in the exhaled breath system to collect 10-100 mL of the exhaled breath.

13. A method to assist in the diagnosis of a neonatal patient developing necrotizing enterocolitis comprising the steps of:
(A) utilizing the hydrogen/carbon dioxide ratio apparatus of claim 1 by:
(i) inserting the exhaled breath system into the neonatal patient's pharynx area over a predetermined time frame to collect a desired quantity of the patient's exhaled breath;
(ii) directing the collected exhaled breath to the air analyzing chamber interconnected to the hydrogen concentration analyzer, the carbon dioxide concentration analyzer, and the air outlet, the collected exhaled breath pushes the air analyzing chamber's residual air through the air outlet;
(iii) measuring the hydrogen concentration of the collected exhaled breath by the hydrogen concentration analyzer and transmitting the hydrogen concentration measurement to the computational analyzer;
(iv) measuring the carbon dioxide concentration of the collected exhaled breath by the carbon dioxide concentration analyzer and transmitting the carbon dioxide concentration measurement to the computational analyzer;
(v) the computational analyzer determines if the collected exhaled breath is an adequate specimen;
(a) if not, the hydrogen concentration measurement and carbon dioxide concentration measurement are discarded;
(b) if so, the computational analyzer (i) calculates the hydrogen/carbon dioxide ratio, (ii) records the hydrogen concentration measurement, carbon dioxide concentration measurement, the hydrogen/carbon dioxide ratio and time of the measurements, and (iii) transmits the recorded hydrogen concentration measurement, carbon dioxide concentration measurement, the hydrogen/carbon dioxide ratio and time of the measurements to the display unit;
(B) displaying the recorded hydrogen concentration measurement, carbon dioxide concentration measurement, the hydrogen/carbon dioxide ratio and time of the measurements on a screen.

14. The method of claim 13, wherein the aspirator is mechanically controlled by the computational analyzer.

15. The method of claim 13, wherein the exhaled breath system has a flow sensor that indicates an exhaled breath.

16. The method of claim 15, wherein the flow sensor interconnects to the computational analyzer and computational analyzer initiates the aspirator to create negative pressure in the exhaled breath system when the flow sensor indicates an exhaled breath.

17. The method of claim 13, wherein the valve is mechanically controlled by the computational analyzer.

18. The method of claim 13, wherein the aspirator creates a sufficient negative pressure in the exhaled breath system to collect 10-100 mL of the exhaled breath.

19. The method of claim 13, wherein the aspirator creates a sufficient negative pressure for 30 to 60 seconds in the exhaled breath system to collect 10-100 mL of the exhaled breath.

20. A kit to assist in the diagnosis of a neonatal patient developing necrotizing enterocolitis comprising:
a hydrogen/carbon dioxide ratio apparatus comprising:
a self-contained, portable hydrogen/carbon dioxide ratio unit having:
an exhaled breath system having a pacifier, an exhaled delivery tube having a length that allows the distal orifice of the exhaled delivery tube to be positioned in the patient's pharynx area, and an aspirator to create a negative pressure in the exhaled deliver tube to collect the patient's exhaled breath into the exhaled delivery tube;
an air analyzing chamber interconnected to a hydrogen concentration analyzer, a carbon dioxide concentration analyzer, and an air outlet, the air analyzing chamber receives the collected patient's exhaled breath so (a) the hydrogen concentration analyzer can measure the amount of hydrogen in the collected air breath and (b) the carbon dioxide concentration analyzer can measure the amount of carbon dioxide in the collected air breath; and
a computational analyzer (a) receives the hydrogen measurement and the carbon dioxide measurement, (b) determines whether the collected air breath is an adequate specimen to retain the hydrogen measurement and the carbon dioxide measurement, (c) only when the collected air breath is adequate, calculates a hydrogen/carbon dioxide ratio, and records the hydrogen measurement, the carbon dioxide measurement, the calculated hydrogen/carbon dioxide ratio and time the measurements occurred; and
a display unit (a) receives the hydrogen measurement, the carbon dioxide measurement, the calculated hydrogen/carbon dioxide ratio and time the measurements occurred, and (b) presents the hydrogen measurement, the carbon dioxide measurement, the calculated hydrogen/carbon dioxide ratio and time the measurements occurred on a screen.
